(19) United States
(12) Patent Application Publication
(10) Pub. No.: US 2004/0171780 A1
(43) Pub. Date: Sep. 2, 2004

(54) Polymer compositions and uses thereof

(76) Inventors: Jo Ann Arceneaux, Marietta, GA (US); Peter Weissman, Woodstock, GA (US)

Correspondence Address: WENDEROTH, LIND & PONACK, L.L.P., 2033 K STREET N. W., SUITE 800, WASHINGTON, DC, 20006-1021, US

(21) Appl. No.: 10/478,253

(22) PCT Filed: May 14, 2002
(86) PCT No.: PCT/EP02/05284

(30) Foreign Application Priority Data
May 25, 2001 (US) 09864211

(51) Int. Cl. C08F 118/02; C07C 69/52

(52) U.S. Cl. 526/319; 558/250; 560/224; 564/152

(57) ABSTRACT

A process comprising the steps of a) reacting in an esterification reaction (i) a plurality of different plyols with; (ii) a plurality of different polycarboxylic acids and/or precursors therefor; in a manner to retain a plurality of free hydroxy groups in the resultant polyester(s) and retain substantial amounts of un-crosslinked polyesters in the resultant product; followed by b) reacting substantially all of the free hydroxy groups present in the resultant mixture of polyesters with an acrylating agent; to form a mixture having a hydroxy (OH) number (measured using ASTM E222-73) of no more than about 100 mg KOH/g. Use of the mixture as a composition for lithographic or flexographic inks.

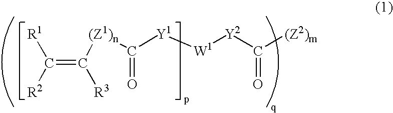

The present invention relates to improved resin compositions which have use in inks for example as a binder and/or grinding vehicle for formulating lithographic and/or flexographic inks.

Previous polyester acrylate resins are known for use as binders but these have disadvantages in terms of cost and/or performance. It has been proposed to blend certain known polyester acrylates to obtain resin compositions having the required balance between cost and performance.

Surprisingly the applicant has discovered that if the reagents (such as polymer precursors) used to prepare certain known polyester acrylates are mixed together and reacted in-situ in a single step and/or single vessel the resultant polymers obtained have improved properties compared to blends of such polymers when made seperately and then combined in a physical blend and/or mixture.

Therefore broadly in accordance with the present invention there is provided a composition comprising at least one compound of Formula 1:

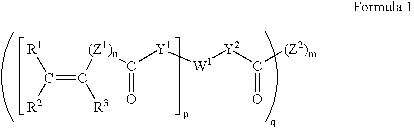

in which

n and/or m independently represent 0 or 1;

p and/or q independently represent an integer from 1 to 5;

R¹ to R³ represent independently, H, optional substuent(s) and/or C₁₋₃₀organo;

Y¹ and y² independently represent oxy, thioxy and/or optionally organo substituted imino;

Z¹ and Z² independently represent a divalent moiety selected from an optional substuent and/or C₁₋₆₀organo; and

W¹ represents a mono to hexavalent C₁₋₃₀organo moiety where each of the Y¹ and the Y² moiet(ies) may be attached to the same and/or different atoms thereon; with the proviso(s) that the composition comprises:

(a) at least three different compounds of Formula 1;

(b) at least one compound of Formula 1 where at least three of the moieties attached to W¹ are different; and/or

(c) further comprises at least two different compounds selected from polyols and/or polyols derivable from a compound of Formula 1.

Further optional features of Formula 1 herein are given below.

Preferably R¹ to R³ are independently H, an optional substituent and/or optionally substituted C₁₋₁₈hydrocarbo.

More preferably R¹ to R³ are independently H and/or optionally substituted C₁₋₁₂hydrocarbo.

Most preferably R¹ to R³ are independently H and/or C₁₋₄hydrocarbo.

Specifically R¹ to R² are both H and R³ is independently H or methyl.

In the examples R¹ is H; R² is H and R³ is H.

Preferably Y¹ and y² are independently —O—, —N(C₁₋₁₂hydrocarbo)—; or —NH—

More preferably Y¹ and y² are independently —O— or —NH—.

In the examples Y¹ is —O— and y² is —O—.

Preferably n is 0; m is 1; p is 3 and/or q is 2.

Preferably Z¹ and Z² are independently optionally substituted C₆₋₆₀hydrocarbo.

More preferably Z¹ is absent and Z² represents C₆₋₁₈arylene or a C₂₀₋₅₀hydrocarbylene, either optionally comprising from one to ten hydrocarbylidene groups.

Most preferably Z¹ is absent and Z² represents C₆₋₁₂arylene or a C₂₀₋₄₀hydrocarbylene comprising four alkenyl groups.

Preferred additional ingredients for and uses of the compositions of the invention comprise any of those given for the acrylic compositions described in U.S. Pat. No. 3,952,032, the contents of which are hereby incorporated by reference.

Preferably W¹ represents tetravalent C₁₋₂₀organo where each of the Y¹ and the y² moie(ties) are attached to different atoms thereon.

More preferably W¹ represents a tetravalent C₁₋₁₅hydrocarbylene optionally comprising one or more oxy groups.

Broadly another aspect of the present invention provides a composition comprising

(I) a plurality of different compounds and/or polymers each compound and/or polymer comprising a plurality, preferably up to five, terminal organo moieties and a central organo moiety; where each terminal moiety comprises at least three, preferably up to six, hydrocarbylidenylcarbonyloxy groups and each terminal moiety is attached to the central moiety via an oxycarbonyl group;

(II) a plurality of different end capping reagents comprising a plurality preferably at least three hydrocarbylidenylcarbonyloxy groups;

where the mixture comprises at least one polymer or compound having different terminal groups thereon.

Broadly another aspect of the present invention provides a process comprising the steps of

(a) reacting in an esterification reaction

(i) a plurality of (preferably two) different polyols (preferably tetraols) with;

(ii) a plurality of (preferably two) different polycarboxylic acids (preferably diacids) and/or precursors therefor (such as anhydrides);

in an manner to retain a plurality of (preferably three) free hydroxy groups in the resultant polyester(s) and retain substantial amounts of uncross-linked polyesters in the resultant product; followed by

(b) reacting substantially all of the free hydroxy groups present in the resultant mixture of polyesters with an acrylating agent (preferably methacrylic acid and/or acrylic acid);

to form mixture comprising: at least one acrylated polyester having terminal ester groups thereon derived from different polyols; and a plurality of acrylated polyols;

the mixture having a hydroxy (OH) number (measured using ASTM E222-73) of no more than about 100 mg, preferably 40 mg KOH/g.

Preferably the product mixture is substantially free of free hydroxy groups.

Preferred polyols for use in the process of the invention comprise any of those listed as suitable for preparing the compositions described in U.S. Pat. No. 3,952,032, for example any two of more of those diols and/or polyols listed on col. 2, from lines 49 to 65 thereof. More preferred polyols may comprise any of the following and/or mixtures thereof:ethylene glycol, propylene glycol, butane-diol, hexane-diol, neopentyl glycol, diethylene glycol, dipropylene glycol, dibutylene glycol, glycerol, trimethylolethane, trimethylolpropane, pentaerythritol, dipentaerythritol, tripentaerythritol, sorbitol, mannitol, inositol, pinitol, quebrachitol and/or α-methylglycoside, most preferably trimethylolpropane and pentaerythritol.

Preferred polycarboxylic acids for use in the process of the invention comprise any of those listed as suitable for preparing the compositions described in U.S. Pat. No. 3,952,032, for example any two of more of those dicarboxylic acids and/or polycarboxylic acids listed on col. 2 from lines 1 to 46 thereof. More preferred polycarboxylic acids may comprise any of the following and/or mixtures thereof:

fatty acids comprising two or three double bonds (such as those available commercially from Emery Industries, Inc under the trade name Empol);

dimers of methyl linoleate and chlorides thereof;

dodecyl-, tetradecyl-, hexadecyl- and octadecyl- succinic acids;

dodecylthiosuccinic acid;

the Diels-Alder addition products of maleic anhydride with conjugated linoleic acid, α or β-eleostearic acid, China wool oil and/or oiticica oil;

the reaction products of maleic anhydride with fatty adds and/or oils comprising one or more non-conjugated double bonds;

the reaction products of maleic, succinic, phthalic, trimellitic, pyromellitic acids and/or anhydrides thereof with ricinoleic acid, higher fatty alcohols, epoxidized higher fatty acids, castor oil and/or epoxidized natural oils;

alkyd resins rich in carboxyl groups obtained by condensing a dimer or trimer acid comprising at least 14 carbon atoms and a diacid having a chain which is shorter than 14 carbon atoms with a polyhydroxylated compound;

the addition product of mercaptobutyric acid with linseed oil, the addition product of thioglycolic acid with linolenic acid; and/or

brassylic acid.

Preferred acrylating agents for use in the process of the invention comprise one or more acrylate moieties as defined herein and/or comprise any of those acrylating agents listed as suitable for preparing the compositions described in U.S. Pat. No. 3,952,032, for example any two of more of those acrylating agents listed from col. 2 line 66 to col. 3, line 20 thereof. More preferred acrylating agents may comprise any of the following and/or mixtures thereof:

the reaction product of 2-hydroxyethyl acrylate, 2-hydroxypropyl acrylate, 4-hydroxybutyl acrylate, 6-hydroxyhexyl acrylate, 8-hydroxy-octyl acrylate, 10-hydroxydecyl acrylate, and/or 12-hydroxydodecyl acrylate with succinic anhydride, maleic anhydride, phthalic anhydride, adipic acid, sebacic acid, and/or itaconic acid;

the reaction product of two moles of a hydroxyalkyl acrylate with one mole of trimellitic anhydride, aconitic acid and/or citric acid; and/or

the reaction product of three moles of a hydroxyalkyl acrylate with one mole of pyromellitic anhydride or acid.

Steps (a) and (b) may be simultaneous or sequential, but preferably are simultaneous

Optionally steps (a) and (b) occur in the same vessel without intermediate isolation and/or purification steps.

Preferably the polyacids also comprise a plurality of unsaturated groups such as an aromatic species (such as benzene ring) and/or from 3 to 5, more preferably 4, ethylenically unsaturated double bonds.

Preferrred polymers are polyester acrylate polymers.

Preferably each polymer having a molecular weight from about 500 daltons to about 3,000 daltons.

Preferably there are two different end cappers.

Preferably the polymer mixtures comprises different polymers comprising separately a plurality (more preferably two) different central moieties, which may be symmetrical or asymmetric and may be substituted by terminal groups along an axis of symmetry so the adduct is symmetric or may be asymmetrically substituted so that the adduct is asymmetric.

Preferably the mixture comprises six polymers in substantially non trace amounts.

Preferably the central organic moiety is a divalent hydrocarbylene or di(hydrocarbylene)oxy moiety more preferably divalent hydrocarbylene, most preferably arylene or poly unsaturated hydrocarbylene.

The terminal groups formed from end capping reagents may comprise from one to six, preferably at least three, acrylate moieties.

Throughout this specification, the term “acrylate moiety” “is used to denote an organo species comprising one or more “hydrocarbylidenylcarbonyloxy” functional groups, analogous and/or derived moieties for example moieties comprising acrylate functionalities and/or derivatives thereof. “Acrylate moieties” may comprise optionally substituted generic α,β-unsaturated acid, ester or other derivatives.

Preferred “acrylate moieties” are represented by Formula A.

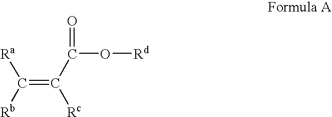

where R^{a}, R^{b}, R^{c} and R^{d} each independently represent H, optionally substituents and/or optionally substituted organo groups; and all suitable isomers thereof, combinations thereof on the same species and/or mixtures thereof.

More preferred moieties of Formula 1 (including isomers and mixtures thereof) are those where R^{a}, R^{b}, R^{c} and R^{d} are independently selected from: H, optional substituents and optionally substituted C₁₋₁₀hydrocarbyl, most preferably H, hydroxy, amino and C₁₆hydrocarbyl, for example H, OH and C₁₋₄alkyl.

Conveniently moieties of Formula A are those where R^{a} and R^{b} are independently H, methyl or OH, and R^{c} is H or CH₃ (i.e. respectively acrylates and methacrylates).

More conveniently acrylate moieties used in the invention are those where R^{a} is OH, R^{b} is CH₃, and R^{c} is H, and/or Formula A represents a tautomer of an acetoacetoxy functional species.

Most convenient acrylate .moieties are selected from: —OCO—CH═CH₂; —OCO—C(CH₃)═CH₂; acetoacetoxy, —OCOCH═C(CH₃)(OH) and all suitable tautomer(s) thereof.

It will be appreciated that any suitable moieties represented by Formula A could also be used as the context dictates.

In the formuale used herein arrows denote a point of attachment (such as a covalent bond) thereto.

The term “independently” as used herein when referring to a list of generic variables (such as n, m and/or p) and/or labelled generic moiet(ies) (such as R¹ to R^{x}) represented in any formual(e) herein denote that those labels may each represent any number(s) and/or moiet(ies) selected from those listed and thus each may be the same as and/or different from each other as appropriate. The term “independently” as also used herein denotes where appropriate that a generic variable and/or moiety represented by single label (such as “n” or R¹) which can occur a plurality of times in the same formula may in each instance therein represent the same and/or different values and/or moeit(ies) selected from those listed, for example where one or more generic subsituent(s) are shown by a single label as repeatedly attached to one or more other moiet(ies) in a formula herein, these may be the same as and/or different from each other in each occurance.

The terms ‘optional substituent’ and/or ‘optionally substituted’ as used herein (unless followed by a list of other substituents) signifies the one or more of following groups (or substitution by these groups): carboxy, sulpho, formyl, hydroxy, amino, imino, nitrilo, mercapto, cyano, nitro, methyl, methoxy and/or combinations thereof. These optional groups include all chemically possible combinations in the same moiety of a plurality (preferably two) of the aforementioned groups (e.g. amino and sulphonyl if directly attached to each other represent a sulphamoyl radical). Preferred optional substituents comprise: carboxy, sulpho, hydroxy, amino, mercapto, cyano, methyl and/or methoxy.

The terms ‘organic substituent’ and “organic group” as used herein (also abbreviated herein to “organo”) denote any univalent or multivalent moiety (optionally attached to one or more other moieties) which comprises one or more carbon atoms and optionally one or more other heteroatoms. Organic groups may comprise organoheteryl groups (also known as organoelement groups) which comprise univalent groups containing carbon, which are thus organic, but which have their free valence at an atom other than carbon (for example organothio groups). Organic groups may alternatively or additionally comprise organyl groups which comprise any organic substituent group, regardless of functional type, having one free valence at a carbon atom. Organic groups may also comprise heterocyclic groups which comprise univalent groups formed by removing a hydrogen atom from any ring atom of a heterocyclic compound: (a cyclic compound having as ring members atoms of at least two different elements, in this case one being carbon). Preferably the non carbon atoms in an organic group herein may be selected from: hydrogen, phosphorus, nitrogen, oxygen silicon and/or sulphur, more preferably from hydrogen, nitrogen, oxygen and/or phosphorous.

Most preferred organic groups comprise one or more of the following carbon containing moieties: alkyl, alkoxy, alkanoyl, carboxy, carbonyl, formyl and/or combinations thereof; optionally in combination with one or more of the following heteroatom containing moieties: oxy, thio, sulphinyl, sulphonyl, amino, imino, nitrilo and/or combinations thereof. Organic groups include all chemically possible combinations in the same moiety of a plurality (preferably two) of the aforementioned carbon containing and/or heteroatom moieties (e.g. alkoxy and carbonyl if directly attached to each other represent an alkoxycarbonyl group).

The term ‘hydrocarbo group’ as used herein is a sub-set of a organic group and denotes any univalent or multivalent moiety (optionally attached to one or more other moieties) which consists of one or more hydrogen atoms and one or more carbon atoms and may comprise saturated, unsaturated and/or aromatic moieties. Hydrocarbo groups may comprise one or more of the following groups. Hydrocarbyl groups comprise univalent groups formed by removing a hydrogen atom from a hydrocarbon. Hydrocarbylene groups comprise divalent groups formed by removing two hydrogen atoms from a hydrocarbon the free valencies of which are not engaged in a double bond. Hydrocarbylidene groups comprise divalent groups (represented by “R₂C═”) formed by removing two hydrogen atoms from the same carbon atom of a hydrocarbon, the free valencies of which are engaged in a double bond. Hydrocarbylidyne groups comprise trivalent groups (represented by “RC═”), formed by removing three hydrogen atoms from the same carbon atom of a hydrocarbon the free valencies of which are engaged in a triple bond. Hydrocarbo groups may also comprise saturated carbon to carbon single bonds; unsaturated double and/or triple carbon to carbon bonds (e.g. alkenyl, and/or alkynyl groups respectively) and/or aromatic groups (e.g. aryl) and where indicated may be substituted with other functional groups.

The term ‘alkyl’ or its equivalent (e.g. ‘alk’) as used herein may be readily replaced, where appropriate and unless the context clearly indicates otherwise, by terms encompassing any other hydrocarbo group such as those described herein (e.g. comprising double bonds, triple bonds, aromatic moieties (such as respectively alkenyl, alkynyl and/or aryl) and/or combinations thereof (e.g. aralkyl) as well as any multivalent hydrocarbo species linking two or more moieties (such as bivalent hydrocarbylene radicals e.g. alkylene).

Any radical group or moiety mentioned herein (e.g. as a substituent) may be a multivalent or a monovalent radical unless otherwise stated or the context clearly indicates otherwise (e.g. a bivalent hydrocarbylene moiety linking two other moieties). However where indicated herein such monovalent or multivalent groups may still also comprise optional substituents. A group which comprises a chain of three or more atoms signifies a group in which the chain wholly or in part may be linear, branched and/or form a ring (including spiro and/or fused rings). The total number of certain atoms is specified for certain substituents for example C_{1-N}organo, signifies a organo moiety comprising from 1 to N carbon atoms. In any of the formulae herein if one or more substituents are not indicated as attached to any particular atom in a moiety (e.g. on a particular position along a chain and/or ring) the substituent may replace any H and/or may be located at any available position on the moiety which is chemically suitable or effective.

Preferably any of the organo groups listed herein comprise from 1 to 60 carbon atoms, more preferably from 1 to 40. It is particularly preferred that the number of carbon atoms in an organo group is from 10 to 20 inclusive.

As used herein chemical terms (other than IUAPC names for specifically identified compounds) which comprise features which are given in parentheses—such as (alkyl)acrylate, (meth)acrylate and/or (co)polymer—denote that that part in parentheses is optional as the context dictates, so for example the term (meth)acrylate denotes both methacrylate and acrylate.

Unless the context clearly indicates otherwise, as used herein plural forms of the terms herein are to be construed as including the singular form and vice versa.

The term “comprising” as used herein will be understood to mean that the list following is non-exhaustive and may or may not include any other additional suitable items, for example one or more further feature(s), component(s), ingredient(s) and/or substituent(s) as appropriate.

The term ‘effective’ and/or “suitable” (for example with reference to the processes, uses, products, materials, formulations, compositions, compounds, monomers, oligomers, polymer precursors and/or polymers used in and/or of the present invention) will be understood to denote utility in any one or more of the following uses and/or applications: preparation and/or use of inks such as those suitable for use in the fields of flexography, lithography and/or graphic arts.

Such utility may be direct where the material has the required properties for the aforementioned uses and/or indirect where the material has use as a synthetic intermediate and/or diagnostic tool in preparing materials of direct utility. As used herein the term “suitable” also denotes that a functional group is compatible with producing an effective product.

The substituents on a repeating unit in any polymers herein may be selected to improve the compatibility of the materials with the polymers and/or resins in which they may be formulated and/or incorporated for the aforementioned uses. Thus, the size and length of the substituents may be selected to optimise the physical entanglement or interlocation with the resin or they may or may not comprise other reactive entities capable of chemically reacting and/or cross-linking with such other resins.

Certain moieties, species, groups, repeat units, compounds, oligomers, polymers, materials, mixtures, compositions and/or formulations which comprise and/or are used in some or all of the invention as described herein may exist as one or more different forms such as any of those in the following non exhaustive list: stereoisomers (such as enantiomers (e.g. E and/or Z forms), diastereoisomers and/or geometric isomers); tautomers (e.g. .keto and/or enol forms), conformers, salts, zwitterions, complexes (such as chelates, clathrates, interstitial compounds, ligand complexes, organometallic complexes, non-stoichiometric complexes, solvates and/or hydrates); isotopically substituted forms, polymeric configurations [such as homo or copolymers, random, graft or block polymers, linear or branched polymers (e.g. star and/or side branched), cross-linked and/or networked polymers, polymers obtainable from di and/or trivalent repeat units, dendrimers, polymers of different tacticity (e.g. isotactic, syndiotactic or atactic polymers)]; polymorphs (such as interstitial forms, crystalline forms and/or amorphous forms), different phases, solid solutions; combinations thereof and/or mixtures thereof. The present invention comprises and/or uses all such forms which are effective.

Compositions of the present invention may be further reacted and/or modified with one or more mono- and/or polyisocyanates such as any of those described in U.S. Pat. No. 3,952,032 col. 6 lines 11 to 64. The urethane compositions so obtained and/or obtainable form a further aspect of the present invention.

In another aspect of the present invention provides a composition and/or formulation obtained and/or obtainable by any of the processes of the invention as described herein.

Compositions of the present invention may be polymerizable by radiation for example as described in U.S. Pat. No. 3,952,032. Compositions of the present invention (whether modified by isocyanates and/or polymerized or not) may comprise further ingredients and/or have further uses to those described analogously for the compositions in U.S. Pat. No. 3,952,032.

A yet further aspect of the invention comprises a method of preparing an ink comprising the steps of mixing a composition and/or formulation of the invention as described herein with a suitable carrier medium and/or grinder.

A still other aspect of the present invention provides an ink obtained and/or obtainable by the preceding method. Preferred inks are those suitable for use in lithographic printing for example for graphic art applications.

A still further aspect of the invention provides use of a composition and/or formulation of the invention to make an ink, preferably a lithographic ink.

A another further aspect of the invention provides use of a ink of the invention (preferably a lithographic ink) to print an article and/or an article so printed.

Other aspects of the present invention may be given in the claims.

The invention will now be illustrated by the following non-limiting examples and tests which are by way of illustration only. Unless otherwise indicated herein all the test results and properties herein were performed using conventional methods well known to those skilled in the art.

In the Examples herein:

Dimer acid denotes an ingredient which is mixture the product of the dimerization of C₁₈ unsaturated mono-basic fatty acids comprising predominately the C₃₆ dimer of linoleic acid of molecular weight 558.59 (i.e. from the C₁₈ double unsaturated fatty acid =11-(1′-carboxyhexyl)-12-butylpentacos-6,9,13,16-tetraen-22-oic acid); the mixture may also comprise a certain (small) amount of the unpolymerized mono-basic acid and/or a trimer acid.

Pentaerythritol is 1,3-dihydoxy-2,2-di(hydroxymethyl)propane.

Di-trimetholylpropane is 3,3,7,7-tetra(hydroxymethyl)-5-oxanonane.

### EXAMPLE 1

(70/30 respective ratio for in situ reaction using polyols for making respectively Comp A & B)

Dimer acid (346.9 g), pentaerythritol (251.22 g), acrylic acid (581.52 g), di-trimethoylpropane (145.52 g), phthalic anhydride (20.62 g), para-toluene sulfonic acid (32.1 g, 65% solution in water), hydroquinone (1.37 g), copper(l)oxide (0.95 g), and hypophosphorous acid (5 g, 50% aqueous solution) were mixed together. Solvent (toluene, 1116 g) was added and the mixture was heated to allow the esterification reaction to proceed. The reaction mixture was allowed to reflux and continue esterification while removing reaction water until the acid value of the reaction mixture was less than 20mgKOH/g. The reaction product was then isolated from the reaction mixture as follows. The esterified product mixture was washed four times at 60° C. with 8% on mass of reaction mixture of a 15% aqueous solution of sodium sulfate. Hydroquinone (0.415 g) and hypophosphorous acid (5 g 50% aqueous solution) were then added and the product mixture was heated to reflux with a constant air sparge. Residual water was removed by azeotropic distillation until substantially all of the water was removed, followed by filtration. The product mixture was concentrated by heating to reflux under reduced pressure with a constant air sparge for typically 16 hours with a final mass temperature of 95° C. The final product was a clear yellow-orange liquid comprised of a complex mixture of polyester acrylate oligomers, esterification by-products, and reagents, and was typically characterized as follows. Viscosity range was typically in the range 21,000-31,0000 cP @25° C., with color typically in the range Gardner 6-8.

### EXAMPLE 2

(50/50 in situ reaction using reagents for making Comp A & B)

Dimer acid (629.68 g), pentaerythritol (334.22 g), acrylic acid (1001.98 g), di-trimethoylpropane (458.66 g), phthalic anhydride (66.3 g), para-toluene sulfonic acid (42.5 g 65% solution in water), hydroquinone (2.992 g), copper(l)oxide (0.697 g), and tri-(nonylphenol)phosphite (2.55 g) were mixed together. Solvent (toluene, 1700 g) was added and the mixture was heated to allow the esterification reaction to proceed. The reaction mixture was allowed to reflux and continue esterification while removing reaction water until the acid value of the reaction mixture was less than 20 mgKOH/g. The product mixture was then isolated from the reaction mixture as follows. The esterified product mixture was washed six times at 65° C. with 10% on mass of reaction mixture of a 15% aqueous solution of sodium sulfate. The product mixture was then heated to reflux with a constant air sparge and residual water removed by azeotropic distillation until substantially all of the water was removed, followed by filtration. Hydroquinone (0.22 g) and tris-(nonylphenol)phosphite (1.7 g)were added and the product mixture was concentrated by heating on a rotovap apparatus under reduced pressure with a constant air sparge. Concentration typically was carried out for 8-10 hours at a bath temperature of about 95° C. Hydroquinone (0.19 g) and tris-(nonylphenol) phosphite (0.68 g) were added at the end of concentration. The final product was a clear orange-brown liquid comprised of a complex mixture of polyester acrylate oligomers, esterification byproducts, and reagents, and was typically characterized as follows. Viscosity range was typically in the range 1,400-2000 cP @60° C., with color typically in the range Gardner 8-12.

Comp A

A known prior art polyester acrylate composition was prepared similarly to as described in Example 12 of U.S. Pat. No. 3,952,032 and was tested as described below.

Comp B

Another prior art polyester acrylate composition was prepared as follows. Dimer acid (475.61 g), di-trimethyolpropane (750.65 g), phthalic anhydride (106.38 g), acrylic acid (643.33 g), paratoluenesulfonic acid (43.40 g 65% solution in water), MeHQ (0.91 g), copper(l)oxide (1.48 g), and tri-(nonylphenol)phosphite (1.36 g) were mixed together. Solvent (toluene, 1,216.91 g) was added and the mixture was heated to allow the esterification reaction to proceed. The reaction mixture was allowed to reflux and continue esterification while removing reaction water until the acid value of the reaction mixture was less than 15 mgKOH/g. The product mixture was then isolated from the reaction mixture as follows. Solvent (toluene, 321.34 g) was added to the reaction mixture and the esterified product mixture was washed three times at 50° C. with 12% on mass of reaction mixture of a 15% aqueous solution of sodium sulfate. MEHQ (0.91 g) and tris(nonylphenol)phosphite (2.71 g) were added. The product mixture was then heated to reflux with a constant air sparge and residual water removed by azeotropic distillation until substantially all of the water was removed, followed by filtration. MEHQ (0.71 g) and tris-(nonylphenol)phosphite (0.70 g) were then added and the product mixture was concentrated by heating to reflux under reduced pressure with a constant air sparge. Concentration typically was carried out for 15 hours with a final mass temperature of about 95° C. Hydroquinone (0.35 g), tris-(nonylphenol)phosphite (1.36 g), and oxalic acid dihydrate (0.41 g) were added and the product was filtered through a 200 μm filter bag. The final product was a orange-brown liquid comprised of a complex mixture of polyester acrylate oligomers, esterification by-products, and reagents. Viscosity was typically in the range 40,000-50,000 cP @ 25° C., with color in the range Gardner 8-12.

TABLE 1
Oligomer viscosities

| Oligomer | Brookfield Viscosity, cP @ 60° C. |
|---|---|
| Comp B | 4610 |
| Comp A | 1910 |
| Example 2 | 1500 |
| 50/50 physical blend | 2190 |
| of Comp A & B | |

Example 2 prepared in situ was based upon a 50:50 mixture of the polyols and polyacids reagent used to prepare the known PEAs of Comp A and Comp B. Example 2 showed an unexpectedly different properties (such as low viscosity) compared to the comparible physical blend of Comp A and Comp B at the same ratio (50:50).

TABLE 2
Comparison of other oligmer properties

| | Viscosity/cP | Tack, 400 RPM | Gardner |
|---|---|---|---|
| Oligomer | @ 25° C. | @ 90° F. | Colour |
| Comp A | 4,500 | 4.9 | 6.5 |
| Comp B | 42,500 | 12.8 | 10.0 |
| Example 2 | 29,100 | 9.3 | 7.0 |

Inks

Conventional lithographic inks were then prepared using as the oligomeric composition (oligomer) the Examples 1 & 2 herein (and for comparison other prior art oligomers). The prepartion was in two stages as follows.

In a first stage, pigment dispersions containing 30% of the conventional pigment Irgalite Rubine L4BD were prepared in a 60/10 blend of the oligomer and the known propoxylated glycol tri-acrylate ingredient for formulating inks available commercially from UCB Chemicals under the trade designation OTA 480. During the preparation of these dispersions the ease of manually adding and blending the pigment into the oligomer and monomer and the appearance of millbase (prior to milling) was assessed as were other properties after three passes through the 3-roll mill.

In the second stage additional conventional amounts of the oligomer, OTA 480 and known conventional photo-initiators were added to each dispersion to prepare a conventional ink formulation—except that inks of the invention comprised the new oligomeric compositions of the invention as exemplified herein. Typical lithographic ink properties such as tack, misting, and reactivity were measured in a conventional manner.

Thus inks of the invention were prepared from Example 2 in this manner and were found to have the following properties: tack 10.6 gm at 1,200 rpm; misting measured by a ΔE of 28; flow of 4.5″/6 min. The inks were found to have a colour strength comparable to prior art inks; an acceptable water window measured on a Duke tester giving a C-type Surland curve. The ink curing reactivity was comparable to prior art inks. The ink of the present invention had acceptable on-press performance; water handling, image quality, and conductivity properties. As used above a prior art ink denotes an otherwise identical ink in which the resin of Example 2 was substituted by a prior art polyester acrylate resin such as Comp A or Comp B. Other inks are compared below:

TABLE 3

| | | Colour density on | | |
|---|---|---|---|---|
| | | substrate | | Reactivity, |
| Example ink | Oligomer | Uncoated | Coated | mJ/cm² 1-400 W/I lamp |
| Comp C | Comp A | 1.05 | 1.31 | 226 |
| Comp D | Comp B | 0.99 | 1.44 | 191 |
| Ex 3 | Example 2 | 1.07 | 1.52 | 186 |

For prior art ink “Comp C” it was relatively easy to establish and maintain a water window. There was no emulsification and the ink produced a good image.

For prior art ink “Comp D” it was moderately easy to establish and maintain water a window. There was slight emulsification and the ink produced a good images.

For the ink of the invention Example 3 it was relatively easy to establish and maintain water window. There was no emulsification and the ink produced a good image. The reactivity of magenta ink made with Example 2 was found to be 86 mJ/cm²

In tables 4 and 5 below: ink tack was measured in a Thwing-Albert electronic inkometer at 1200 RPM, 90° F. for 3 minutes; Comp E was a 50:50 physical blend of the prior art oligomeric mixtures Comp A & Comp B.

TABLE 4
Inks

| | Ink Tack | Misting | Reactivity, mJ/cm² |
|---|---|---|---|
| Oligomer | g (average) | ΔE (avg) | 1-400 watts/in lamp |
| Comp B | 12.5 | 7.7 | 327 |
| Example 1 | 10.8 | 7.3 | 274 |
| Comp E | 8.1 | 9.1 | 640 |
| | | | (in 2 passes) |

Thus lithographic inks of the invention demonstrate good color development; acceptable ink tack and misting properties; and good printing capabilities. Furthermore inks of the invention can perform similarly to prior art inks on the press, have much lower color, and costs less to manufacture than the best prior art inks.

It has also been observed that inks made from physical blends of prior art components (especially 50:50 blends of Comp A and Comp B) compare unfavourably with the (in situ reacted) mixtures of the present invention as the blends tend to exhibit high misting and low tack values. It is also considerably more difficult to add pigment to physical blends compared to the mixtures of the present invention. Yet the mixtures of the invention had comparable reactivities to a reactive oligomer (Comp B) while the physical blend of Comp A and Comp B was much less reactive (e.g. required two passes at the lowest conveyor speed to achieve a cure).

Thus the in situ reacted mixtures of the present invention exhibited improved properties and a good balance between the properties of a blend and the best component thereof having more of the advantages of both with fewer of the disadvantages of either.

Thus without wishing to be bound by any theory it is believed that the in situ esterification used to prepare Examples 1 & 2 of the present invention generates a product mixture with more components than the prior art compositions Comp A or Comp B or mixtures thereof. Thus the improved properties of Example 1 & 2 may be due to these additional components generated for example the presence of effective amounts of more than one polyol, as such components are not believed to be present in either Comp A, Comp B or an physical blend thereof.

1. A composition comprising at least one compound of Formula 1: 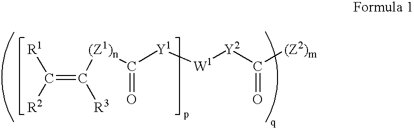
in which
n and/or m independently represent 0 or 1;
and/or q independently represent an integer from 1 to 5;
R¹ to R³ represent independently, H, optional substituent(s) and/or C₁₋₃₀organo;
Y¹ and y² independently represent oxy, thioxy and/or optionally organo substituted imino;
Z¹ and Z² independently represent a divalent moiety selected from an optional substituent and/or C₁₋₆₀organo; and
W¹ represents a mono to hexavalent C₁₋₃₀organo moiety where each of the Y¹ and the Y² moiet(ies) may be attached to the same and/or different atoms thereon;
with the proviso(s) that the composition comprises:
(a) at least three different compounds of Formula 1;
(b) at least one compound of Formula 1 where at least three of the moieties attached to W¹ are different; and/or
(c) further comprises at least two different polyols derivable from a compound of Formula 1 and/or a component thereof.

2. A composition according to claim 1, in which in Formula 1 R¹ to R³ are independently H, an optional substituent and/or optionally substituted C₁₋₁₈hydrocarbo.

3. A composition according to claim 2, in which R¹ to R³ are independently H and/or optionally substituted C₁₋₁₂hydrocarbo.

4. A composition according to claim 3, in which R¹ to R³ are independently H and/or C₁₋₄hydrocarbo.

5. A composition according to claim 4, in which R¹ to R² are both H and R³ is independently H or methyl.

6. A composition according to claim 5, in which R¹ is H; R² is H and R³ is H.

7. A composition according to claim 1, in which in Formula 1 Y¹ and y² are independently —O—, —N(C₁₋₁₂hydrocarbo)-; or —NH—

8. A composition according to claim 9, in which Y¹ and Y² are independently —O— or —NH—.

9. A composition according to claim 10, in which Y¹ and Y² are both —O—.

10. A composition according to claim 1, in which in Formula 1, n is 0; m is 1, p is 3 and q is 2.

11. A composition according to claim 1, in which in Formula 1, Z¹ and Z² are independently optionally substituted C₆₋₆₀hydrocarbo.

12. A composition according to claim 11, in which Z¹ is absent and Z² represents C₆₋₁₈arylene or a C₂₀₋₆₀hydrocarbylene, either optionally comprising from one to ten hydrocarbylidene groups.

13. A composition according to claim 12, in which Z¹ is absent and Z² represents C₆₋₁₂arylene or a C₂₀₋₄₀hydrocarbylene comprising four alkenyl groups.

14. A composition according to claim 1, in which in Formula 1, W¹ represents tetravalent C₁₋₂₀organo where each of the Y¹ and the Y² moie(ties) are attached to different atoms thereon.

15. A composition according to claim 14, in which W¹ represents a tetravalent C₁₋₁₅hydrocarbylene optionally comprising one or more oxy groups.

16. A composition comprising
(I) a plurality of different compounds and/or polymers each compound and/or polymer comprising two terminal organo moieties and a central organo moiety; where each terminal moiety comprises three hydrocarbylidenylcarbonyloxy groups and each terminal moiety is attached to the central moiety via an oxycarbonyl group;
(III) a plurality of different end capping reagents comprising at least three hydrocarbylidenylcarbonyloxy groups;
where the mixture comprises at least one polymer or compound having different terminal groups thereon.

17. A process comprising the steps of
(a) reacting in an esterification reaction
(i) a plurality of different polyols with;
(iii) a plurality of different polycarboxylic acids and/or precursors therefor;
in an manner to retain a plurality of (preferably three) free hydroxy groups in the resultant polyester(s) and retain substantial amounts of un-cross-linked polyesters in the resultant product; followed by
(b) reacting substantially all of the free hydroxy groups present in the resultant mixture of polyesters with an acrylating agent;
to form mixture comprising: at least one acrylated polyester having terminal ester groups thereon derived from different polyols; and a plurality of acrylated polyols;
the mixture having a hydroxy (OH) number (measured using ASTM E222-73) of no more than about 100 mg KOH/g, optionally the mixture being substantially free of hydroxy comprising species.

18. A composition obtained and/or obtainable by a process as claimed in claim 17.

19. A method of preparing an ink comprising the steps of mixing a composition as claim in any preceding claim with a suitable carrier medium.

20. An ink obtained and/or obtainable by a process as claimed in claim 19.
